(19) United States
(12) Patent Application Publication
(10) Pub. No.: US 2024/0341998 A1
(43) Pub. Date: Oct. 17, 2024

(54) FLUID COLLECTION DEVICES HAVING ONE OR MORE OF MOLDED, EXTRUDED, AND/OR FILM PORTIONS, AND RELATED SYSTEMS AND METHODS

(71) Applicant: PUREWICK CORPORATION, Covington, GA (US)

(72) Inventors: Alexandra Julian, Elmhurst, IL (US); Joseph Ranalletta, Greenville, NC (US); Gonghao Wang, Cary, NC (US); John Hayes, Raleigh, NC (US)

(21) Appl. No.: 18/294,403

(22) PCT Filed: Aug. 1, 2022
(86) PCT No.: PCT/US2022/039022

§ 371 Date: Feb. 1, 2024

Related U.S. Application Data
(60) Provisional application No. 63/228,258, filed on Aug. 2, 2021.

(51) Int. Cl. A61F 5/451

(52) U.S. Cl. CPC A61F 5/451

(57) ABSTRACT

Examples relate to fluid collection devices, and related systems and methods. In some embodiments, a fluid collection device includes an elongated fluid impermeable barrier and an elongated body. The fluid collection device includes at least one of the fluid impermeable barrier being an injection molded fluid impermeable barrier, the body including at least an extruded support, the fluid impermeable barrier including one or more regions of film welded together, the fluid impermeable barrier including a die cut foam fluid impermeable barrier, or the fluid impermeable barrier including a plastic tube welded at the proximal end region and the distal end region.

### CROSS-REFERENCE TO RELATED APPLICATIONS

This application claims priority to U.S. Provisional Patent Application No. 63/228,258 filed on Aug. 2, 2021, the disclosure of which is incorporated herein, in its entirety, by this reference.

### BACKGROUND

An individual may have limited or impaired mobility such that typical urination processes are challenging or impossible. For example, the individual may have surgery or a disability that impairs mobility. In another example, the individual may have restricted travel conditions such as those experienced by pilots, drivers, and workers in hazardous areas. Additionally, fluid collection from the individual may be needed for monitoring purposes or clinical testing.

Bed pans and urinary catheters, such as a Foley catheter, may be used to address some of these circumstances. However, bed pans and urinary catheters have several problems associated therewith. For example, bed pans may be prone to discomfort, spills, and other hygiene issues. Urinary catheters be may be uncomfortable, painful, and may cause urinary tract infections.

Thus, users and manufacturers of fluid collection devices continue to seek new and improved devices, systems, and methods to collect urine.

### SUMMARY

Embodiments disclosed herein are related to fluid collection devices, and related systems and methods. In an embodiment, a fluid collection device includes an elongated fluid impermeable barrier and an elongated body. The elongated fluid impermeable barrier includes a distal end region and a proximal end region at least partially defining a chamber, an opening in fluid communication with the chamber and positioned on the fluid collection device to receive urine discharged from a urethra at least proximate to the opening, and an aperture sized and dimensioned to have urine drawn therethrough by a vacuum source. The elongated body is positioned at least partially within the chamber and extends at least partially across the opening, the elongated body being at least partially fluid permeable. The fluid collection device may include at least one of the fluid impermeable barrier being an injection molded fluid impermeable barrier, the body including at least an extruded support, the fluid impermeable barrier including one or more regions of film welded together, the fluid impermeable barrier including a die cut foam fluid impermeable barrier, or the fluid impermeable barrier including a plastic tube welded at the proximal end region and the distal end region.

In an embodiment, a method of manufacturing a fluid collection device includes, with at least one of injection molding, welding, or heat shrinking, forming an elongated fluid impermeable barrier having a distal end region and a proximal end region at least partially defining a chamber, an opening in fluid communication with the chamber and positioned on the fluid collection device to receive urine discharged from a urethra at least proximate to the opening, and an aperture sized and dimensioned to have urine drawn therethrough by a vacuum source. The method also may include positioning an elongated body that is at least partially fluid permeable at least partially within the chamber and extending at least partially across the opening.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1 is a block diagram of a fluid collection system, according to an embodiment.

FIG. 2A is a front isometric view of a fluid collection device, according to an embodiment.

FIG. 2B is a longitudinal cross-sectional view of the fluid collection device of FIG. 2A.

FIG. 2C is a transverse cross-sectional view of the fluid collection device of FIG. 2A.

FIG. 2D is an exploded isometric view of the fluid collection device of FIG. 2A.

FIG. 3A is a front isometric view of a fluid collection device, according to an embodiment.

FIG. 3B is a rear isometric view of the fluid collection device of FIG. 3A.

FIG. 3C is an exploded view of the fluid collection device of FIG. 3A.

FIG. 4A is a front isometric view of a fluid collection device, according to an embodiment.

FIG. 4B is a longitudinal cross-sectional view of the fluid collection device of FIG. 4A.

FIG. 4C is an exploded view of the fluid collection device of FIG. 4A.

FIG. 5A is a front isometric view of a fluid collection device, according to an embodiment.

FIG. 5B is a longitudinal cross-sectional view of the fluid collection device of FIG. 5A.

FIG. 5C is an exploded view of the fluid collection device of FIG. 5A.

FIG. 6 is a flow diagram of a method for collecting fluid, according to an embodiment.

### DETAILED DESCRIPTION

Embodiments disclosed herein are related to fluid collection devices, and related systems and methods of manufacture. The devices and systems disclosed herein are configured to collect fluids from an individual. The fluids collected by the fluid collection devices may include at least one of urine, vaginal discharge, penile discharge, reproductive fluids, blood, sweat, wound exudate, or other bodily fluids. At least one, some, or all embodiments of fluid collection devices described herein result in the technical effect of improved methods of manufacturing and assembly of fluid collection devices, and/or a technical effect of improved fluid collection devices resulting from these methods of manufacturing and assembly. For example, at least one, some, or all embodiments described herein result in the technical effect of more efficient and repeatable manufacturing and assembly of fluid collection devices, thereby providing repeatability and reducing the cost and/or time for assembly of the fluid collection device(s).

In various embodiments, the fluid collection devices may be formed by methods of manufacture and/or assembly that include one or more of: an extruded support (e.g. core) inserted into a molded fluid impermeable barrier (e.g., housing); a form-fill-seal process that may include forming the impermeable barrier, filling the chamber with a body that is at least partially fluid permeable, and sealing the fluid collection device; films bonded or welded together to form a fluid impermeable barrier; and/or a body that is at least partially fluid permeable positioned and sealed within a molded tube.

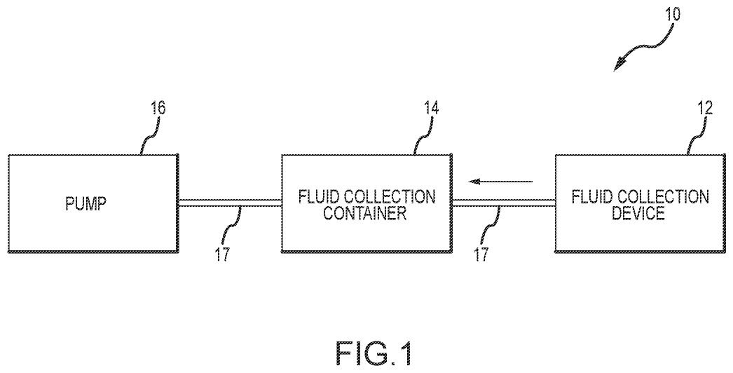

FIG. 1 is a block diagram of a fluid collection system 10, according to an embodiment. The fluid collection system 10 may be included in embodiments of fluid collection systems described herein. The system 10 includes a fluid collection device 12 (e.g., any of the fluid collection devices disclosed herein), a urine collection container 14, and a pump 16 (or vacuum source). The fluid collection device 12, the urine collection container 14, and the pump 16 may be fluidly coupled to each other via one or more conduits 17. For example, the fluid collection device 12 may be operably coupled to one or more of the urine collection container 14 or the pump 16 via the conduit 17. In some embodiments, the pump 16 may be secured directly to the urine collection container 14. Fluid (e.g., urine or other bodily fluids) collected in the fluid collection device 12 may be removed from the fluid collection device 12 via the conduit 17 secured to the fluid collection device 12. Suction force may be introduced into the chamber of the fluid collection device 12 via the inlet of the conduit 17 responsive to suction (e.g., vacuum) force applied at the outlet of the conduit 17.

The suction force may be applied to the outlet of the conduit 17 by the pump 16 either directly or indirectly. The suction force may be applied indirectly via the urine collection container 14. For example, the outlet of the conduit 17 may be disposed within or fluidly coupled to an interior region of the urine collection container 14 and an additional conduit 17 may extend from the urine collection container 14 to the pump 16. Accordingly, the pump 16 may apply suction to the fluid collection device 12 via the urine collection container 14. The suction force may be applied directly via the pump 16. For example, the outlet of the conduit 17 may be disposed within the pump 16. An additional conduit 17 may extend from the pump 16 to a point outside of the fluid collection device 12, such as to the urine collection container 14. In such examples, the pump 16 may be disposed between the fluid collection device 12 and the urine collection container 14.

The urine collection container 14 is sized and shaped to retain a fluid therein. The urine collection container 14 may include a bag (e.g., drainage bag), a bottle, a canister, or a cup (e.g., collection jar), or any other enclosed container for storing bodily fluid(s) such as urine. In some examples, the conduit 17 may extend from the fluid collection device 12 and attach to the urine collection container 14 at a first point therein. An additional conduit 17 may attach to the urine collection container 14 at a second point thereon and may extend and attach to the pump 16. Accordingly, a vacuum (e.g., suction) may be drawn through fluid collection device 12 via the urine collection container 14. Fluid, such as urine, may be drained from the fluid collection device 12 using the pump 16.

The pump 16 may include one or more of a manual vacuum pump, and electric vacuum pump, a diaphragm pump, a centrifugal pump, a displacement pump, a magnetically driven pump, a peristaltic pump, or any pump configured to produce a vacuum. The pump 16 may provide a vacuum or suction to remove fluid from the fluid collection device 12. In some examples, the pump 16 may be powered by one or more of a power cord (e.g., connected to a power socket), one or more batteries, or even manual power (e.g., a hand operated vacuum pump). In some examples, the pump 16 may be sized and shaped to fit outside of, on, or within the fluid collection device 12. For example, the pump 16 may include one or more miniaturized pumps or one or more micro pumps. The vacuum sources disclosed herein may include one or more of a switch, a button, a plug, a remote, or any other device suitable to activate the pump 16.

Fluid collection devices described herein may include a fluid impermeable barrier and a body that is at least partially fluid permeable. The fluid impermeable barrier may be elongated and have a distal end region and a proximal end region, and may at least partially define a chamber, an opening in fluid communication with the chamber and positioned on the fluid collection device to receive urine discharged from a urethra proximate to the opening, and an aperture in the proximal end region sized and dimensioned to have urine drawn therethrough by a vacuum source. The body may be elongated and positioned at least partially within the chamber to extend at least partially across the opening. Embodiments of the fluid collection devices described herein may include at least one of the fluid impermeable barrier being an injection molded fluid impermeable barrier, the body including at least an extruded support, the fluid impermeable barrier including one or more regions of film welded together, the fluid impermeable barrier including a die cut foam fluid impermeable barrier, or the fluid impermeable barrier including a plastic tube welded at the proximal end region and the distal end region.

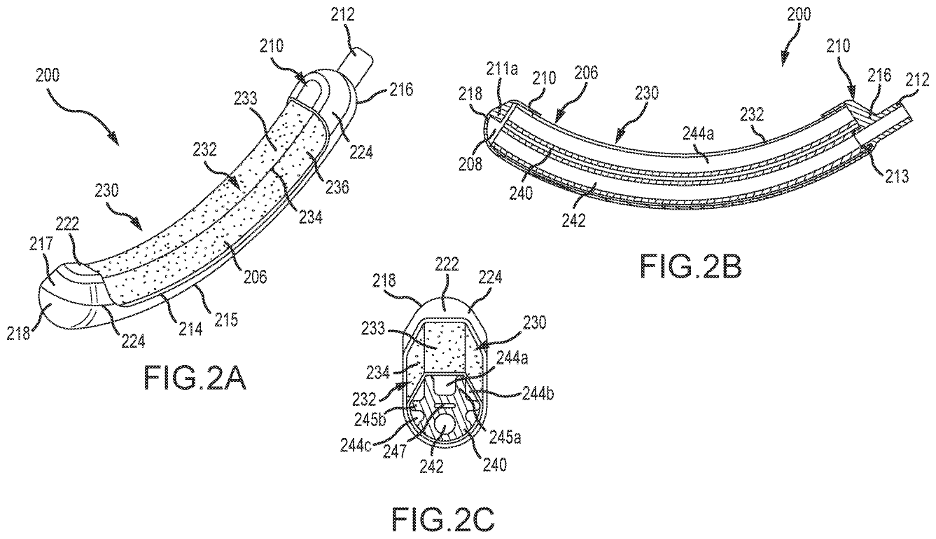

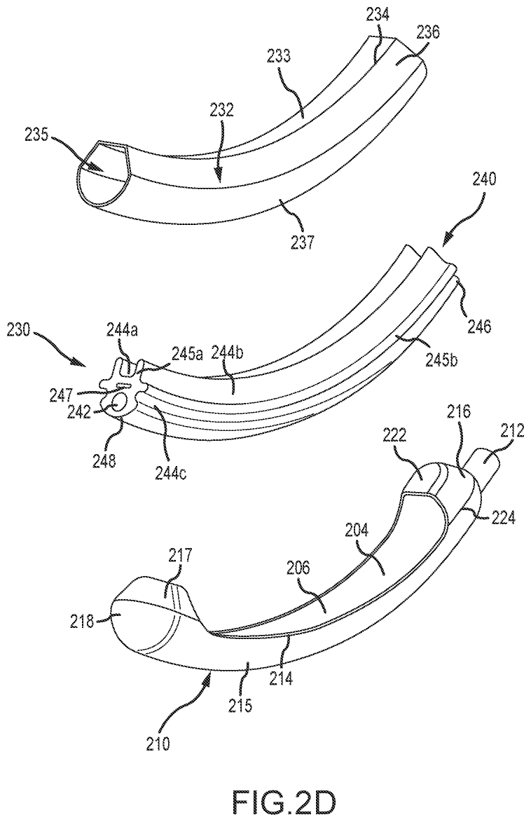

In some embodiments, a fluid collection device may include an extruded support including spun plastic and surrounded at least partially by an outer wicking layer and inserted into a housing of a fluid impermeable barrier. Turning to FIGS. 2A-2D, a fluid collection device 200 may include a fluid impermeable barrier 210 and a body 230 that is at least partially fluid permeable. The fluid impermeable barrier 210 may be elongated and include a distal region 218, a proximal end region 216, an aperture 212 or port positioned at the proximal end region 216 and sized and dimensioned to urine drawn therethrough by a vacuum source. The fluid impermeable barrier 210 may include a back side 215 and a front side 217. The front side 217 is positioned to be oriented towards the user when the fluid collection device 200 is in use. In some embodiments, the fluid impermeable barrier 210 may at least partially define a chamber 204 (shown in FIG. 2D) and an opening 206 on the front side 217 positioned on the fluid collection device 200 to receive urine discharged form a urethra that is at least proximate (e.g., adjacent) to the opening 206. In some embodiments, an inward border or edge 214 of the fluid impermeable barrier 202 defines the opening 206. The edge 214 may include two or more opposing arced portions, the arced portions following the outer circumference or periphery of the fluid impermeable barrier 210.

The fluid impermeable barrier 210 also may at least partially define a reservoir 208 (e.g., sump) in the chamber 204 at the distal end region 218 of the fluid impermeable barrier 210. The reservoir 208 may be positioned for fluid collection in the fluid collection device 200 before withdraw from the fluid collection device 200 through a channel 242 in body 230. In some embodiments, the reservoir 208 may be an at least partially (e.g., entirely) unoccupied portion of the chamber 204 and is at least partially (e.g., entirely) void of other material. In some embodiments, the reservoir 208 may be substantially filled with one or more fluid permeable materials described herein. In some embodiments, the reservoir 208 is defined at least partially by a terminating end the body 230 (shown in FIG. 2B) and the distal end region 218 of the fluid impermeable barrier 210.

In some embodiments, the fluid impermeable barrier 210 may include one or more protrusions 211a positioned within the chamber 204 at the distal end region 218 to space at least a portion of the channel 242 and/or the slots 244a, 244b from the distal end region 218 of the fluid impermeable barrier 210 effective to allow fluid communication between channel 242 and/or the slots 244a, 244b. For example, the fluid impermeable barrier 210 may include a protrusion 211a at the distal end region 218 protruding into the reservoir 208. The protrusion 211a may be sized smaller and/or thinner than the channel 242 and/or the slots 244a, 244b such that if the body 230 is pushed too far into the chamber 204, the protrusion 211a may contact only a portion (e.g., a front portion) of the body 230, but also space a portion of the channel 242 and/or the slots 244a, 244b from the distal end region 218 of the fluid impermeable barrier 210. This space between the channel 242 and/or the slots 244a, 244b and the distal end region 218 of the fluid impermeable barrier 210 allows fluid to still be pulled into the channel 242 even when the protrusion 211a is contacting a portion of the body 230. The protrusion 211a, then, results in the technical effect of preventing the channel 242 from being blocked or plugged if the body 230 is pushed too far into the chamber 204. In some embodiments, the distal end region 218 includes an alignment feature (not shown) extending partially into the channel 242 of the extruded support 240. The alignment feature may be shaped complementary to at least a portion of the channel 242 and may include one or more slits, openings, apertures, or perforations configured to provide fluid communication between the channel 242 and the reservoir 208. In some embodiments, at least one of the alignment feature or the channel 242 includes one or more tabs or stops positioned on the alignment feature and/or the channel 242 to prevent the alignment feature from being inserted too far into the channel 242. The one or more tabs or stops, then, may result in the technical effect of insuring fluid communication between the channel 242 and the reservoir is maintained.

In some embodiments, the distal end region 218 includes wire sockets for a U-shaped wire shape memory polymer or metal and/or a socket for a malleable strip. For example, the strip may include an elongated strip that extends through at least the distal end region 218 into the passage 247. In some embodiments, the proximal end region 216 at least partially defines the aperture 212. The aperture 212 may be defined by a portion (e.g., external port) of the proximal end region 216 extending outward from the proximal end region 216 and configured to detachably secure to a conduit (e.g., tube). The proximal end region 216 also may include an internal port 213 extending partially into the channel 242 in the proximal end region of the extruded support 240.

The fluid impermeable barrier 210 may be elongated and have a cross-sectional shape complementary to the periphery of the body 230. For example, in FIG. 2C, the shape back side 215 of the fluid impermeable barrier 215 may be generally arced, curved, or semi-circular. The front side 217 may include a substantially planar face 222 and sidewalls 224 angling between the back side 215 and the face 222. Other embodiments of the fluid impermeable barrier 210 may include other geometrical shapes and configurations. The fluid impermeable barrier 210 may be formed of any suitable fluid impermeable materials, such as a fluid impermeable polymer (e.g., silicone, polypropylene, polyethylene, polyethylene terephthalate, a polycarbonate, etc.), polyurethane films, thermoplastic elastomer (TPE), rubber, thermoplastic polyurethane (TPU), another suitable material, or combinations thereof. The fluid impermeable barrier 210 is flexible, thereby enabling the fluid collection device 200 to bend or curve when positioned against the body of a wearer. Example fluid impermeable barriers may include, but are not limited to, a fluid impermeable barrier including at least one of Versaflex CL 2000X TPE, Dynaflex G6713 TPE, or Silpuran 6000/05 A/B silicone. In an embodiment, the fluid impermeable barrier 210 may be air permeable. In such an embodiment, the fluid impermeable barrier 210 may be formed of a hydrophobic material that defines a plurality of pores. In an embodiment, one or more portions of at least the outer surface of the fluid impermeable barrier 210 may be formed from a soft and/or smooth material, thereby reducing chaffing.

In some embodiments, the fluid impermeable barrier 210 may be molded, such as injection molded. For example, the fluid impermeable barrier 210 may be injection molded from one or more of silicone or a polymer such as TPE or TPU. The fluid impermeable barrier 210 may be injection molded as one component. In some embodiments, the fluid impermeable barrier 210 may be injection molded to be curved or arced longitudinally. In at least one, some, or all embodiments, injection molding the fluid impermeable barrier 210 results in the technical effect of reducing the cost of manufacturing the fluid impermeable barrier 210 and the fluid collection device 200. For example, injection molding the fluid impermeable barrier 210 according to at least one, some, or all embodiments provides the technical effect of reducing the material cost and providing a faster cycle time for manufacturing the fluid impermeable barrier 210. Injection molding the fluid impermeable barrier 210 also results in the technical advantage of repeatability and consistency in at least one, some, or all embodiments. Injection molding the fluid impermeable barrier 210 also results in the technical advantage of incorporating features described herein into a single body in at least one, some, or all embodiments.

The elongated body 230 may be at least partially fluid permeable and has a portion of the body 230 that is not covered by the fluid impermeable barrier 210 and is positioned on the fluid collection device 200 to receive urine discharged from a urethra at least proximate to the portion of the body 230 that is not covered by the fluid impermeable barrier 210. For example, the body 230 may include an extruded support 240 and a fluid permeable membrane 232 extending at least partially (e.g., entirely) across the opening 206. In some embodiments, the extruded support 240 may include a foamed or soft material extrusion, such as a closed cell foam extrusion. The extruded support 240 may be generally fluid impermeable and support the fluid permeable membrane 232 of the body 230.

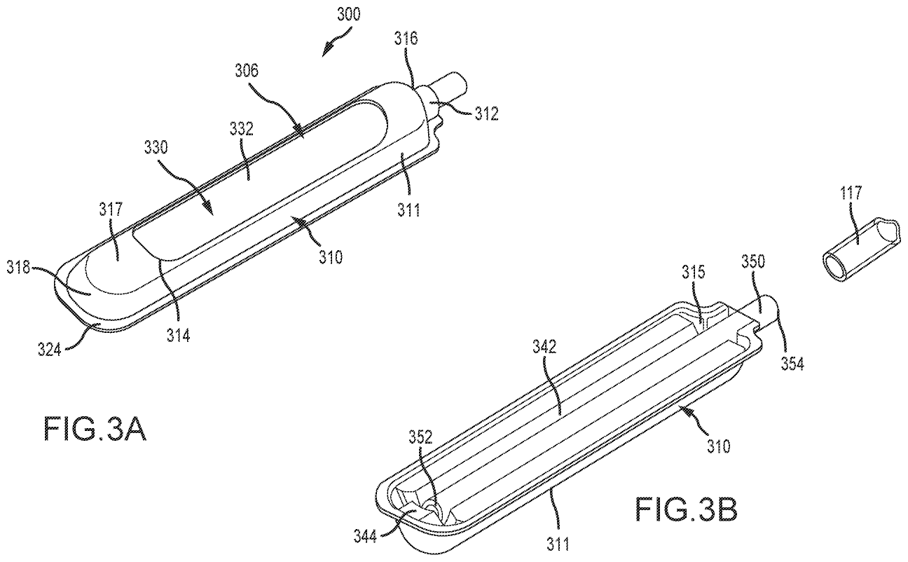

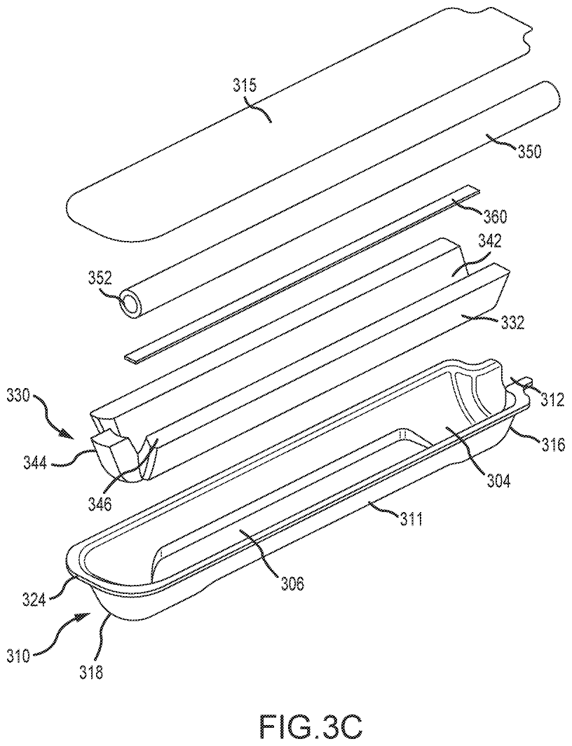

In some embodiments, the extruded support 240 includes a distal end region 248, a proximal end region 246, and at least one slot 244a extending longitudinally on the extruded support 240. More particularly, the extruded support 240 may include a rear side 248 and a front side including two side slots 244b extending longitudinally on the front side of the extruded support with the slot 244a being positioned between the two side slots 244b. The extruded support 240 may include a plurality of longitudinal ridges 245a at least partially defining the slots 244a, 244b. For example, the slot 244a may be defined at least partially by the longitudinal ridges 245a. The side slots 244b may be defined at least partially by one of the longitudinal ridges 245a and a side longitudinal ridge 245b. In some embodiments, the extruded support 240 includes one or more rear slots 244c at least partially defined by the side longitudinal ridge 245b. The extruded support 240 may include a channel 242 extending longitudinally therethrough. The channel 242 may be completely enclosed within the extruded support 240 such that the extruded support 240 surrounds and/or defines the channel 242. For example, the channel 242 may include an O- or D-shaped channel extending longitudinally through the extruded support 240 from the distal end region 248 to the proximal end region 246. The extruded support 240 also may include one or more lumens 247 extending longitudinally at partially through the extruded support 240 and positioned to receive a shape memory polymer or metal (similar to the strip 360 shown in FIG. 3C). For example, the extruded support 240 may include a lumen 247 sized and dimensioned to receive a malleable polymer or metal and/or two lumens sized and dimensioned to receive arms or prongs of a U-shaped malleable polymer or metal wire.

The fluid permeable membrane 232 and the fluid permeable support 240 also can wick and/or allow transport of the fluid generally towards the reservoir 208. The fluid permeable membrane 232 can include any material that can wick the fluid. For example, the fluid permeable membrane can include fabric, such as a gauze (e.g., a silk, linen, polymer based materials such as polyester, or cotton gauze), nylon (such as a spun nylon fibers), another soft fabric (e.g., jersey knit fabric or the like), or another smooth fabric (e.g., rayon, satin, or the like). Forming the fluid permeable membrane from gauze, soft fabric, and/or smooth fabric can reduce chaffing caused by the fluid collection device 200. Other embodiments of fluid permeable membranes and fluid permeable supports are disclosed in U.S. patent application Ser. No. 15/612,325 filed on Jun. 2, 2017; U.S. patent application Ser. No. 15/260,103 filed on Sep. 8, 2016; U.S. patent application Ser. No. 15/611,587 filed on Jun. 1, 2017; PCT Patent Application No. PCT/US19/29608, filed on Apr. 29, 2019, the disclosure of each of which is incorporated herein, in its entirety, by this reference. In some embodiments, the body 230 includes a fluid permeable support, such as a porous spun nylon fiber structure and the fluid permeable wicking membrane 232 including gauze at least partially enclosing the spun nylon fiber structure. For example, the body 230 may include a gauze or other wicking fabric positioned to contact the skin of the user through the opening 206.

In some embodiments, the fluid permeable membrane 232 is secured or securable the extruded support 240 such that a portion of the fluid permeable membrane 232 extends across the multiple slots 244a, 244b on the front side of the extruded support 240. The portion of the fluid membrane 232 that extends across at least some (e.g., all) of the multiple slots 244a-c may include the portion of the body 230 that is not covered by the fluid impermeable barrier 210 and extends across the opening 206 to receive urine discharged from a urethra. In some embodiments, the fluid permeable membrane 232 may wrap around the periphery of the extruded support 240. For example, the fluid permeable membrane 232 may be generally tubular when wrapped around the periphery of the extruded support 240. The ridges 245a, 254b on the extruded support 240 may support the fluid permeable membrane 232 such that at least some (e.g., all) of the multiple slots 244a-c remain open and void of material (other than urine and/or a support member). When wrapped around the extruded support 240, the portion of the fluid permeable membrane 232 not covered by the fluid impermeable barrier 210 may include a substantially planar center region 233 covering the slot 244a and two substantially planar side regions 236 covering the slots 244b and angling towards the substantially planar center region 233.

In use, the fluid collection device 200 may be positioned with the fluid permeable membrane 232 that covers at least the multiple slots 244a, 44b at least proximate (e.g., adjacent) to the urethra of the user. The ridges 245a, 245b on the extruded support 240 may support the fluid permeable membrane 232 such that the multiple slots 244a, 244b remain open and void of material (other than urine). With a vacuum creating a negative pressure on the fluid collection device 200, urine discharged on the fluid permeable membrane 232 may be drawn or otherwise enter one or more of the slots 244a, 244b and flow to the reservoir 208. The vacuum may draw the urine in the reservoir 208, into the channel 242, through the aperture 212, and out of the fluid collection device 200.

In some embodiments, the fluid collection device may be formed through a form-fill-seal process. For example, a fluid impermeable barrier (or membrane) may be formed before a body that is at least partially fluid permeable is sealed within a chamber formed by the fluid impermeable barrier. Turning now to FIGS. 3A-3C, in some embodiments, a fluid collection device 300 may include a fluid impermeable barrier 310 having a first portion 311 and a second portion 315. The first portion 311 of the fluid impermeable barrier 310 may include an injection molded fluid impermeable barrier and/or a die cut foam including a front region 317 at least partially defining a chamber 304 and an opening 306. The first portion 311 of the fluid impermeable barrier 310 may include fluid impermeable material described above in relation to the fluid impermeable barrier 210. In some embodiments, the first portion 311 of the fluid impermeable barrier 310 may include a polystyrene or other foam material formed through injection molding and/or die cutting. In some embodiments, the first portion 311 of the fluid impermeable barrier 310 may include a polyvinyl chloride (PVC) material, such as a PVC bag-like material. In some embodiments, the fluid impermeable barrier 310 may include any material that may be radio frequency (or high frequency) welded to other materials, such as one or more of polyethylene terephthalate, polyethylene terephthalate glycol, PVC, thermoplastic polyurethanes, open celled polyurethanes, low density polyethylene, and/or ethylene-vinyl acetate. In some embodiments, the first portion 311 of the fluid impermeable barrier 310 may be formed via an extrusion process that forms a sheet or film that is then formed into the partial cylinder shape of the first portion 311 of the fluid impermeable barrier 310 through one or more of heat, pressure, and/or vacuum conditions. This process of manufacturing the first portion 311 of the fluid impermeable barrier 310 may be a continuous process, resulting in the technical effect of more efficient manufacturing and production of the fluid collection device 300 in at least one, some, or all embodiments.

The second portion 315 of the fluid impermeable barrier 310 may include a substantially planar sheet 315 or film bonded or welded to the first portion 311 of the fluid impermeable barrier 310 distal to the opening 310. For example, the sheet 315 may be radio frequency or ultrasonically welded to the first portion 311 of the fluid impermeable barrier 310. In some embodiments, the sheet 315 may be secured to the first portion 311 of the fluid impermeable barrier 310 with bonding and/or an adhesive. The sheet 315 may extend from the distal end region 318 to the proximal end region 316 of the fluid impermeable barrier 310 and at least partially define the chamber 304 and the aperture 312. For example, the sheet 315 and the first portion 311 of the fluid impermeable barrier 310 may define the aperture 312 sized for a conduit 350 to extend therethrough into the chamber 304. In some embodiments, the sheet 315 may be clear, transparent, or translucent. The sheet 315 also may include instructions for use of the fluid collection device 300 and/or product information of the fluid collection device 300, either directly on the sheet 315 or a label secured to the sheet 315.

The fluid collection device 300 also may include a body positioned within the chamber 304. The body may include a laminated and/or die cut fluid permeable body 330. The fluid permeable body 330 can be configured to wick and/or allow transport of fluid away from the opening 306 towards a reservoir in the chamber 304 and/or an inlet 352 of the conduit 350. The fluid permeable body 330 can include any material that can wick and/or allow transport of the fluid. The permeable properties referred to herein can be wicking, capillary action, diffusion, or other similar properties or processes, and are referred to herein as “permeable” and/or “wicking.” Such “wicking” or other physical properties may exclude absorption into the fluid permeable body 330, such as not include adsorption of the bodily fluids into the fluid permeable body 330. Put another way, substantially no absorption or solubility of the bodily fluids into the material may take place after the material is exposed to the bodily fluids and removed from the bodily fluids for a time. While no absorption or solubility is desired, the term “substantially no absorption” may allow for nominal amounts of absorption and/or solubility of the bodily fluids into the fluid permeable body 330 (e.g., absorbency), such as less than about 30 wt % of the dry weight of the fluid permeable body 330, less than about 20 wt %, less than about 10 wt %, less than about 7 wt %, less than about 5 wt %, less than about 3 wt %, less than about 2 wt %, less than about 1 wt %, or less than about 0.5 wt % of the dry weight of the fluid permeable body 330. In an embodiment, the fluid permeable body 330 may include at least one absorbent or adsorbent material.

The fluid permeable body 330 can include a one-way fluid movement fabric. As such, the fluid permeable body 330 can remove fluid from the area around the urethra, wound, and/or skin, thereby leaving the area dry. The fluid permeable body 330 can enable the fluid to flow generally towards the reservoir and/or the inlet 352. The fluid permeable body 330 can include a porous or fibrous material, such as hydrophilic polyolefin. In some embodiments, the fluid permeable body 330 consists of or consists essentially of a porous or fibrous material, such as hydrophilic polyolefin. Examples of polyolefin that can be used in the fluid permeable body 330 include, but are not limited to, polyethylene, polypropylene, polyisobutylene, ethylene propylene rubber, ethylene propylene diene monomer, or combinations thereof. Moreover, the fluid permeable body 330 can be manufactured according to various manufacturing methods, such as laminating, die-cutting, molding, extrusion, and/or sintering. In some embodiments, the fluid permeable body 330 may include open or closed cell foam extrusion. The fluid permeable body 330 can include varying densities or dimensions.

In some embodiments, the fluid permeable body 330 can include two or more layers of fluid permeable materials. For example, the fluid permeable body 330 can include a fluid permeable membrane defining an outer surface 332 of the fluid permeable body 330 that extends across the opening 306 of the front region 317 of the fluid impermeable barrier 310. The fluid permeable membrane may cover at least a portion of a fluid permeable support, with both the fluid permeable membrane and the fluid permeable support being disposed at least partially in the chamber 304. The fluid permeable membrane and the fluid permeable support can be configured to wick any fluid away from the opening 306, thereby preventing the fluid from escaping the chamber 304 through the opening 306 and promoting removal of the fluid from the chamber 304 through the conduit 350. The permeable properties referred to herein can be wicking, capillary action, diffusion, or other similar properties or processes, and are referred to herein as “permeable” and/or “wicking.” The fluid permeable membrane and/or the fluid permeable support may include any of the materials described above in relation to the fluid permeable membrane 232.

In some embodiments, the body 330 may include a two sidewalls 346 angling or curving towards one another and defining a slot 342 oriented towards the sheet 315 and aligned with the aperture 312. The slot 342 may be sized to position at least a portion of the conduit 350 therein. The body 330 also may include a distal portion 344 that at least partially terminates the slot 342. The distal portion 344 of the body 330 may extend from an intersection of the sidewalls 346 towards the sheet 315.

The fluid collection device 300 also may include the conduit 350 (e.g., tube) removably or fixedly secured at least partially within the chamber 304. The conduit 350 may extend through the aperture 312 into the chamber 304 and may be positioned at least partially in the slot 342 of the body 330. The conduit 350 also may include an inlet 352 positioned proximate to the distal portion 344 of the body 330 and/or the distal end region 318 of the fluid impermeable barrier 310. In some embodiments, the inlet 352 of the conduit 350 is adjacent to and/or abuts the distal portion 344 of the body 330. The conduit 350 may provide fluid communication between an interior region of the chamber 304 and a fluid storage container (not shown) and/or a vacuum source (not shown). For example, the conduit 350 may directly or indirectly fluidly couple the interior region of the chamber 304 with the fluid storage container and/or the portable vacuum source. In some embodiments, the conduit 350 extends through the aperture 312 and includes an outlet 354 positioned outside the chamber 304. The outlet 354 may be configured to connect with an additional conduit 117 in fluid communication with a vacuum source effective to provide fluid communication between the conduit 350 and the additional conduit 117. The fluid collection device 300 also may include a seal formed by one or more of the first portion 311, the sheet 315, and/or separate seal that conforms around the conduit 350 proximate to the aperture 312 effective prevent leaking from the chamber 304 of the fluid collection device 300.

In some embodiments, the fluid collection device 330 also includes a support member 360. The support member 360 may be detachably or fixedly secured or securable to at least one of the first portion 311 of the fluid impermeable barrier 310, the body 330, the conduit 350, and/or the sheet 315. For example, the support member 360 may be positioned in the slot 342 between the conduit 350 and the body 330, embedded in the body 330, and/or extending through the body 330. The support member 360 may include a shape memory polymer or metal, such as a strip or a U-shaped bracket or staple. Suitable shape memory materials are composed to adopt an intermediate or permanent shape in response to a stimuli. The stimuli may include an external physical force (e.g., bending force), heat, electrical bias, or a magnetic field. While the term “shape memory” is used to describe some of the “shape memory materials” herein, it should be understood that, in some examples, the material modified by the term “shape memory” may not necessarily need to return to a preselected shape upon application of a stimuli, as understood as the classical definition of the “shape memory material.” Rather, at least some of the shape memory materials herein may simply hold a selected shape when bent, set, or cured into a specific shape and/or when cooled in a specific shape, regardless of the stimuli applied thereto after. The shape memory materials may be returned to the original shape or changed to a new shape by application of stimuli. For example, a metal wire bent to a first shape may be utilized as the shape memory material, whereinafter the metal wire may be modified to a second shape via physical force applied thereto or via heating.

In an embodiment, the shape memory material may include metal, such as an elemental metal, an alloy, or shape memory alloy. Suitable shape memory metals may include standard steels, stainless steel, carbon alloy steel, head treated steel, aluminum, silver, copper, iron, nickel, zinc, tin, beryllium, or the like. Suitable shape memory alloys may include stainless steel; galvanized steel; aluminum alloys; nickel-titanium alloys, such as Nitinol, Ni—Ti—Cu, Ni—Ti, Co, or the like; copper-based alloys such as Cu—Zn—Al, Cu—Al—Ni, Cu—Al—Sn, or the like; Co—Cr—Ni—Mo alloys (e.g., Elgiloy®) or the like; or any other alloy having shape memory characteristics. As explained above, the shape memory metals or alloys may merely be metals or alloys that may be shaped to a selected configuration. In some examples, the shape memory metals or alloys may return to a primary shape when an external stimuli is applied thereto. In some examples, the outer surface of the shape memory metal may be coated with a polymer, anodized, passivated, or otherwise treated to prevent corrosion.

Shape memory polymers (“SMPs”) may include polyurethane-based SMPs such as a copolymer (e.g., copolyester, polyurethane, polyetherester, etc.) including blocks of one or more of poly(ε-caprolactone), polyethyleneterephthalate (PET), polyethyleneoxide (PEO), polyethylene glycol (PEG), polystyrene, polymethylmethacrylate (PMMA), Polybutylmethacrylate (PBMA), poly(N,N-butadiene), poly(N-methyl-N-oxazoline), polytetrahydrofuran, or poly(butylene terephthalate); thermoplastic polymers such as polyether ether ketone (PEEK), nylon, acetal, polytetrafluoroethylene (PTFE), polysulphone, or the like; Polynorbonene; other deformable polymers; or any other shape memory polymer.

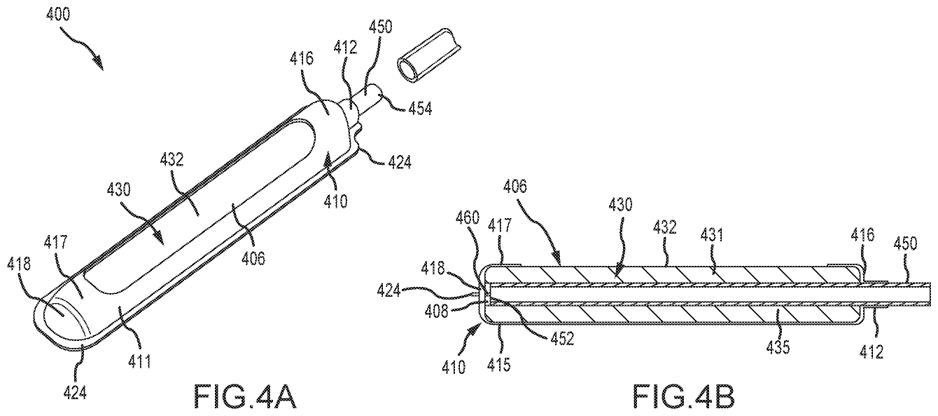

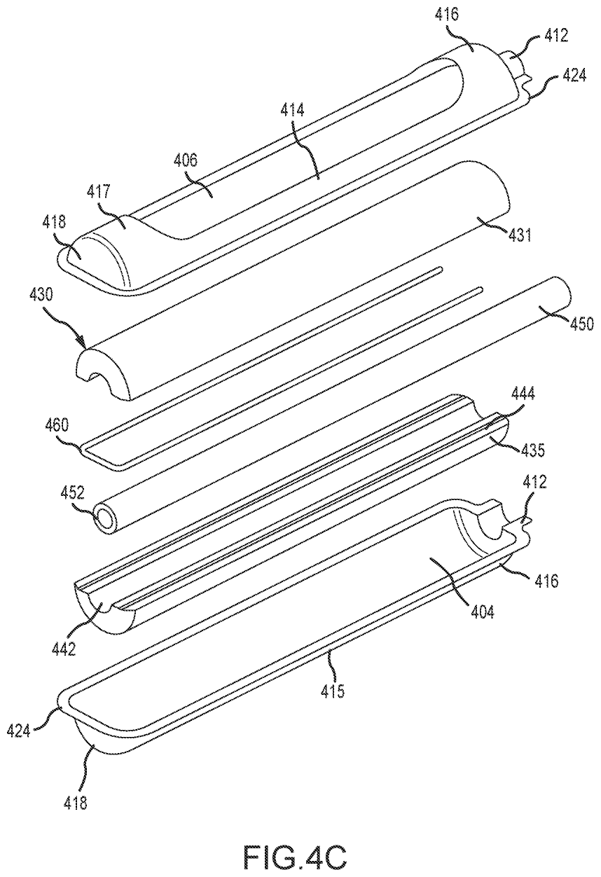

Turning now to FIGS. 4A-4C, in some embodiments, a fluid collection device 400 may include a fluid impermeable barrier 410 having a first (e.g., front) portion 411 and a second (e.g., rear) portion 415. The first portion 411 of the fluid impermeable barrier 410 may include any aspect of the first portion 311 of the fluid impermeable barrier 310 in the fluid collection device 300. For example, the first portion 411 of the fluid impermeable barrier 410 may include an injection molded fluid impermeable barrier and/or a die cut foam including a front region 417 at least partially defining a chamber 404 and an opening 406. The first portion 411 of the fluid impermeable barrier 410 may include any fluid impermeable material described above in relation to the fluid impermeable barrier 210 and/or any materials and manufacturing of the first portion 311 of the fluid impermeable barrier 310. In some embodiments, the first portion 411 of the fluid impermeable barrier 410 may include a plastic film.

The second portion 415 of the fluid impermeable barrier 410 may be arced or curved such that the first portion 411 and the second portion 415 together form a generally cylindrical shape. For example, the second portion 415 may include a cross-sectional shape that is half- or semi-circular, and the first portion 411 also may include a cross-sectional shape that is half- or semi-circular. In some embodiments, the fluid impermeable barrier 410 may include other geometrical shapes and configurations. The first portion 411 and the second portion 415 may together at least partially define the chamber 404 of the fluid collection device 400. The first portion 411 and the second portion 415 may together at least partially define the aperture 412 at the proximal end region 416 of the fluid impermeable barrier 416.

The second portion 415 of the fluid impermeable barrier 410 may include any fluid impermeable material described above in relation to the fluid impermeable barrier 210 and/or any materials and manufacturing of the first portion 311 of the fluid impermeable barrier 310. In some embodiments, the second portion 415 of the fluid impermeable barrier 410 may include a plastic film. The second portion 415 may be bonded or welded to the first portion 411 of the fluid impermeable barrier 410 on a seam 424 that extends from the aperture 412 to the distal end region 418 of the fluid impermeable barrier 410. For example, the seam 424 from the aperture 412 to the distal end region 418 on a first side of the fluid impermeable barrier 410, across the distal end 418 opposite to the aperture 412, and then from the distal end region 418 to the aperture on a second side of the fluid impermeable barrier 410 with the opening 406 positioned between the first side and second side of the fluid impermeable barrier 410. In some embodiments, the second portion 415 of the fluid impermeable barrier 410 may be radio frequency or ultrasonically welded to the first portion 411 of the fluid impermeable barrier 410 on the seam 424. In some embodiments, the second portion 415 may be secured to the first portion 411 of the fluid impermeable barrier 410 with bonding and/or an adhesive on the seam 424.

The fluid impermeable barrier 410 also may at least partially define a reservoir 408 (e.g., sump) in the chamber 404 at the distal end region 418 of the fluid impermeable barrier 410. The reservoir 408 may be positioned for fluid collection in the fluid collection device 400 before the fluid is withdrawn from the fluid collection device 400 through the conduit 450 and/or the channel 442. In some embodiments, the reservoir 408 may be an at least partially (e.g., entirely) unoccupied portion of the chamber 404 and is at least partially (e.g., entirely) void of other material. In some embodiments, the reservoir 408 is substantially filled with one or more of the fluid permeable materials described herein. In some embodiments, the reservoir 408 is defined at least partially by the fluid permeable body 430 and the distal end region 418 of the fluid impermeable barrier 410. In some embodiments, the reservoir 408 is filled at least partially (e.g., entirely) with the fluid permeable body 430.

The fluid collection device 400 also may include a body positioned within the chamber 404. The body may include a laminated and/or die cut fluid permeable body 430. The fluid permeable body 430 can be configured to wick and/or allow transport of fluid away from the opening 406 towards a reservoir in the chamber 404 and/or an inlet 452 of the conduit 450. The fluid permeable body 430 can include any material and/or configuration that can wick and/or allow transport of the fluid. For example, the fluid permeable body 430 may include any material or configuration described above in relation to the fluid permeable body 330. In some embodiments, the fluid permeable body 430 can include two or more layers of fluid permeable materials. For example, the fluid permeable body 430 can include a fluid permeable membrane defining an outer surface 432 of the fluid permeable body 430 that extends across the opening 406 of the front region 417 of the fluid impermeable barrier 410. The fluid permeable membrane may cover at least a portion of a fluid permeable support, with both the fluid permeable membrane and the fluid permeable support being disposed at least partially in the chamber 404.

In some embodiments, the fluid permeable body 430 may include a first portion 431 and a second portion 435 that together form a generally cylindrical fluid permeable body 430 or other shape that is complementary the shape of the chamber 404 and/or the fluid impermeable barrier 410. The first portion 411 and the second portion 415 may be die-cut to a predetermined shape. In some embodiments, the first portion 431 of the fluid permeable body 430 may be shaped complementary to the first portion 411 of the fluid impermeable barrier 410, and the second portion 435 of the fluid permeable body 430 may be shaped complementary to the second portion 415 of the fluid impermeable barrier 410. The fluid permeable body 430 may include a channel 442 defined at least partially by one or more (e.g., both) of the first portion 431 and the second portion 435. The channel 442 may longitudinally extend through at least some (e.g., all) of the fluid permeable body 430 and may be sized to position at least a portion of the conduit 450 therein. In some embodiments, the fluid permeable body 330 may include an integrally formed fluid permeable body 330 defining the channel 442, rather than separate first and second portions 431, 435. When the fluid permeable body 430 is positioned in the chamber 404, the channel 442 may be substantially aligned with the aperture 412.

In some embodiments, at least one (e.g. both) of the first portion 431 and the second portion 435 of the fluid permeable body 430 include a slot 444 extending longitudinally at least partially through the fluid permeable body 430 and sized to receive a support member 460. The fluid collection device 400 also may include the support member 460, which may include any of the materials of the support member 360. The support member 460 may include an elongated U-shaped support member or staple having two arms extending longitudinally through the fluid permeable body 430. In some embodiments, at least a portion of the support member 460 may include a wire or other element that is positioned to extend across the channel 442 without covering all of the channel 442. This portion of the support member 460 results in the technical effect of preventing the inlet 452 of the conduit 450 from contacting the distal end region 418 of the fluid impermeable barrier 410 and becoming clogged by the fluid impermeable barrier 410 in at least one, some, or all embodiments. Instead, this portion of the support member 460 extending across the channel 442 results in the technical effect of ensuring fluid communication between the fluid permeable body 330 and the conduit 450 and/or the channel 442 is maintained in at least one, some, or all embodiments.

The fluid collection device 400 also may include the conduit 450 (e.g., tube) removably or fixedly secured or securable at least partially within the chamber 404. The conduit 450 may extend through the aperture 412 into the chamber 404 and may be positioned at least partially in the channel 442 of the fluid permeable body 430. The conduit 450 also may include an inlet 452 positioned proximate to the distal portion of the body 430 and/or the distal end region 418 of the fluid impermeable barrier 410. The inlet 452 is spaced from the distal end region 418 of the fluid impermeable barrier 410 according to an embodiment. For example, the support member 460 may include a wire extending across the channel 442 and the inlet 452 that prevents the inlet 452 from contacting or abutting the fluid impermeable barrier 418. In some embodiments, the fluid permeable body 430 may include a tab in the channel 442 that prevents the inlet 452 from contacting the distal end region 418 of the fluid impermeable barrier 410 such that the inlet 452 does not become clogged by the fluid impermeable barrier 410. The conduit 450 may provide fluid communication between an interior region of the chamber 404 and a fluid storage container (not shown) or a vacuum source (not shown). For example, the conduit 450 may directly or indirectly fluidly couple the interior region of the chamber 404 with the fluid storage container or the portable vacuum source. In some embodiments, the conduit 450 extends through the aperture 412 and includes an outlet 454 positioned outside the chamber 404. The outlet 454 may be configured to connect with an additional conduit 117 in fluid communication with a vacuum source effective to provide fluid communication between the conduit 450 and the additional conduit 117. The fluid collection device 400 also may include a seal formed by one or more of the first portion 411, the second portion 415, and/or a separate seal that conforms around the conduit 450 proximate to the aperture 412 resulting in the technical effect of preventing leaking from the chamber 404 of the fluid collection device 400 in at least one, some, or all embodiments.

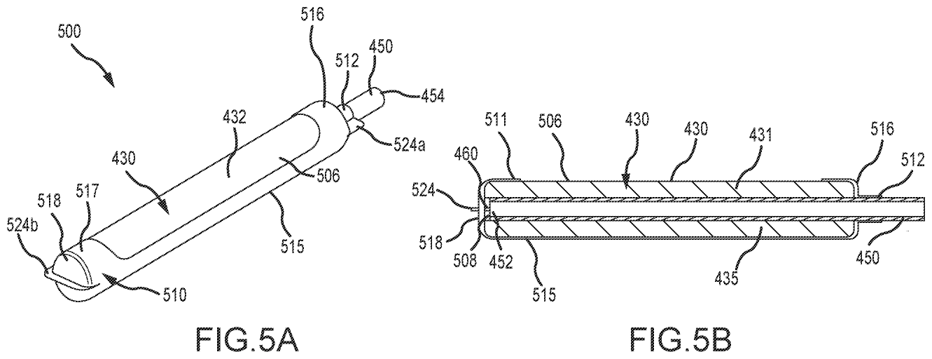

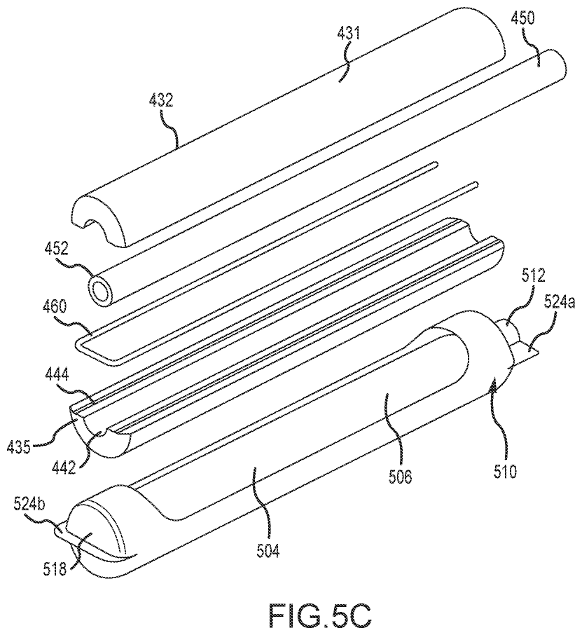

Turning now to FIGS. 5A-5C, a fluid collection device 500 may include a fluid impermeable barrier 510 and the fluid permeable body 430. Unless otherwise noted, the fluid collection device 500 may include any aspect of the fluid collection devices 200, 300, 400. For example, the fluid collection device 500 may include the fluid permeable body 430 and the support member 460 of the fluid collection device 400. The fluid impermeable barrier 510 may include any materials described above in relation to the fluid impermeable barriers 210, 310, 410.

In some embodiments, the fluid impermeable barrier 510 is generally tubular and/or cylindrical or other geometrical shapes and configurations. The fluid impermeable barrier 510 may include a distal end region 518 and a proximal end region 516 defining an aperture 512. The fluid impermeable barrier 510 also may at least partially define a chamber 504 and include a front side 511 at least partially defining an opening 506 in fluid communication with the chamber 504. A back or rear side 515 of the fluid impermeable barrier 510 opposite the front side 511 may be devoid of any opening. The fluid impermeable barrier 410 may include any fluid impermeable material described above in relation to the fluid impermeable barriers 210, 310, 410 and/or any materials and manufacturing of the first portions 311, 411 of the fluid impermeable barriers 310, 410. In some embodiments, the fluid impermeable barrier 510 may include an injection molded fluid impermeable barrier and/or a die cut foam. In some embodiments, the fluid impermeable barrier 510 may include a plastic film or other plastic material formed (e.g., extruded or molded) as tube. In some embodiments, the fluid impermeable barrier 510 may be heat shrunk around the fluid permeable 430.

In some embodiments, the fluid impermeable barrier 510 is sealed at the distal end region 518 and the proximal end region 516. For example, the fluid impermeable barrier 510 may be bonded or welded on a proximal seam 524a at the proximal end region 516 and a distal seam 524b at the distal end region 518 of the fluid impermeable barrier 510. In some embodiments, the fluid impermeable barrier 510 may be radio frequency or ultrasonically welded on the seams 524. In some embodiments, the fluid impermeable barrier 510 may be sealed at the seams 524 with bonding and/or an adhesive. At least a portion of the fluid impermeable barrier 510 between the distal seam 524b and the proximal seam 524a may be void of a seam.

The fluid impermeable barrier 510 also may at least partially define a reservoir 508 (e.g., sump) in the chamber 504 at the distal end region 518 of the fluid impermeable barrier 410. The reservoir 508 may be positioned for fluid collection in the fluid collection device 500 before withdraw from the fluid collection device 500 through the conduit 550 and/or the channel 542. In some embodiments, the reservoir 508 may be an at least partially (e.g., entirely) unoccupied portion of the chamber 504 and is at least partially (e.g., entirely) void of other material. In some embodiments, the reservoir 508 is defined at least partially by the fluid permeable body 430 and the distal end region 418 of the fluid impermeable barrier 410. In some embodiments, the reservoir 508 is filled at least partially (e.g., entirely) with the fluid permeable body 430.

In some embodiments, the fluid collection device 500 may include the fluid impermeable barrier 510 and a body including a fluid permeable membrane wrapped around or secured to an extruded support. For example, the fluid collection device 500 may include a body similar to or the same as the body 230 of the fluid collection device 200, such as an extruded support 240 including at least one slot 244a covered by the fluid permeable membrane 232 and providing fluid communication between the channel 242 in the support 240 and the opening 506 in the fluid impermeable barrier 510. The fluid impermeable barrier 510 and/or the extruded support 240 may be shaped generally complementary to one other. The conduit 550 may extend only partially into the channel 242 at the proximal end region of the fluid impermeable barrier.

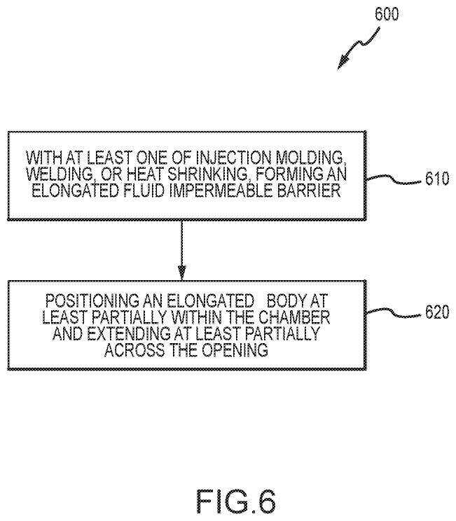

FIG. 6 is a flow diagram of a method 600 of manufacturing a fluid collection device, according to an embodiment. The method 600 includes an act 610 of forming, with at least one of injection molding, welding, and/or heat shrinking, an elongated fluid impermeable barrier. The fluid impermeable barrier formed in the act 610 may include a distal end region and a proximal end region and may at least partially define a chamber, an opening in fluid communication with the chamber and positioned on the fluid collection device to receive urine discharged from a urethra proximate to the opening, and an aperture sized and dimensioned to have urine drawn therethrough by a vacuum source. The method 600 also includes an act 620 of positioning an elongated body that is at least partially fluid permeable at least partially within the chamber and extending at least partially across the opening.

In some embodiments, the method 600 further includes an act of extruding a support of the body before positioning the body at least partially within the chamber. Extruding the support may include extruding the support to include a channel extending longitudinally therethrough, a front side including multiple front slots extending longitudinally along the front side of the extruded support, and a rear side including multiple rear slots. In these and other embodiments, the method 600 may include wrapping a fluid permeable membrane around the extruded support to form the body that is at least partially fluid permeable, the fluid permeable membrane covering the multiple front slots and the multiple rear slots. The method 600 also may include inserting a shape memory polymer or metal longitudinally into the extruded support. The multiple front slots may include two side slots and an intermediate slot positioned between the two slots on the extruded support and extruding the support includes extruding the support to include the rear side to be generally arced.

In these and other embodiments, the act 610 may include forming the elongated fluid impermeable barrier with injection molding. More particularly, forming the elongated fluid impermeable barrier may include forming the fluid impermeable barrier to include a rear side arced complementary to the rear side of the extruded support, a generally planar front side covering at least some of the intermediate slot, and two intermediate sides angled between the rear side and the front side. Each of the two intermediate sides may cover at least some of a different one of the two side slots when the body is positioned in the chamber of the fluid impermeable barrier. In some embodiments, forming the fluid impermeable barrier includes forming the fluid impermeable barrier to include an internal port at the proximal end region in fluid communication with the aperture and an external port at the proximal end region at least partially defining the aperture. In these and other embodiments, the act 620 may include positioning the internal port at least partially in the channel of the extruded support.

In some embodiments, the act 610 of forming an elongated fluid impermeable barrier may include forming the elongated fluid impermeable barrier with injection molding or die cutting foam to include a front region at least partially defining the chamber and the opening. The act 620 may include positioning the elongated body at least partially within the chamber with a slot on the elongated body oriented away from the opening on the front region of the fluid impermeable barrier and aligned with the aperture. In these and other embodiments, the method 600 also may include, after positioning the fluid permeable body at least partially within the chamber, bonding or welding a fluid impermeable sheet to the front region distal to the opening with the sheet extending from the distal end region to the proximal end region of the fluid impermeable barrier and at least partially defining the chamber and the aperture.

In some embodiments, the method 600 may include an act of laminating and/or die cutting the body. In some embodiments, the method 600 may include an act of positioning a conduit to extend through the aperture into the chamber at least partially in the slot of the body with an inlet of proximate to the distal end region of the fluid impermeable barrier. In some embodiments, the method 600 may include positioning a shape memory polymer or metal proximate to the body.

In some embodiments, the act 610 include includes welding a front region of plastic film to a rear region of plastic film to at least partially define the chamber with the front region defining the opening. The act 620 may include positioning the body within the chamber with a channel defined by the body aligned with the aperture. In these and other embodiments, the method 600 may include an act of inserting a conduit through the aperture and at least a portion of the channel to position an inlet on the conduit in the chamber proximate to the distal end region of the fluid impermeable barrier. The method also may include an act of positioning a shape memory polymer or metal having two arms longitudinally in the body with the channel positioned between the two arms, such as positioning the shape memory polymer or metal having two arms longitudinally in the body between two separate die-cut fluid permeable portions of the body.

In some embodiments, welding a front region of plastic film to a rear region of plastic film may include welding the front region of plastic film to the rear region of plastic film on a seam that extends from the aperture to the distal end region. In some embodiments, welding a front region of plastic film to a rear region of plastic film includes welding the front region of plastic film to the rear region of plastic film on a distal seam and a proximal seam, without welding at least a portion of the fluid impermeable barrier between the distal seam and the proximal seam.

The method also may include an act of extruding a support of the body and wrapping a fluid permeable membrane of the body around the extruded support. In some embodiments, the method also may include an act of comprising heat shrinking the fluid impermeable barrier around at least a portion of the body.

The acts of the method 600 described above are for illustrative purposes. For example, the acts of the method 600 can be performed in different orders, split into multiple acts, modified, supplemented, or combined. In an embodiment, one or more of the acts of the method 600 can be omitted from the method 600. Any of the acts of the method 600 can include manufacture of any of the fluid collection devices and systems disclosed herein.

As used herein, the term “about” or “substantially” refers to an allowable variance of the term modified by “about” or “substantially” by ±10% or ±5%. Further, the terms “less than,” “or less,” “greater than,” “more than,” or “or more” include, as an endpoint, the value that is modified by the terms “less than,” “or less,” “greater than,” “more than,” or “or more.”

While various aspects and embodiments have been disclosed herein, other aspects and embodiments are contemplated. The various aspects and embodiments disclosed herein are for purposes of illustration and are not intended to be limiting.

1. A fluid collection device, comprising:
an elongated fluid impermeable barrier having a distal end region and a proximal end region and at least partially defining a chamber, an section in fluid communication with the chamber and positioned on the fluid collection device to receive urine discharged from a urethra at least proximate to the section, and an aperture sized and dimensioned to have urine drawn therethrough by a vacuum source; and
an elongated body positioned at least partially within the chamber and extending at least partially across the section, the elongated body being at least partially fluid permeable,
wherein the fluid impermeable barrier includes a first portion at least partially defining the section and a second portion, the elongated body being positioned in the chamber between the first portion and the second portion such that the first portion protrudes outward away from the second portion, and the second portion of the fluid impermeable barrier including a film bonded or welded to the first portion of the fluid impermeable barrier.

2-6. (canceled)

7. The fluid collection device of claim 1, wherein:
the fluid impermeable barrier includes:
the first portion including an injection molded fluid impermeable barrier or a die cut foam including a front region at least partially defining the chamber and the section;
the second portion including a substantially planar sheet of film bonded or welded to the first portion of the fluid impermeable barrier opposite to the section, the sheet extending from the distal end region to the proximal end region of the fluid impermeable barrier and at least partially defining the chamber and the aperture; and
the body is positioned within the chamber and includes a slot oriented towards the sheet and aligned with the aperture.

8. The fluid collection device of claim 7, wherein the body includes a laminated and/or die cut body.

9. The fluid collection device of claim 7, further comprising a conduit extending through the aperture into the chamber and being positioned at least partially in the slot of the body, the conduit having an inlet positioned proximate to the distal end region of the fluid impermeable barrier.

10. The fluid collection device of claim 7, further comprising a shape memory polymer or metal positioned proximate to the body.

11. The fluid collection device of claim 1, wherein:
the first portion of the fluid impermeable barrier includes a front region of plastic film defining the section;
the second portion of the fluid impermeable barrier include a rear region of plastic film welded to the first portion to at least partially define the chamber; and
the body is generally cylindrical and defines a channel extending longitudinally through at least a portion of the body and aligned with the aperture.

12. The fluid collection device of claim 11, further comprising a conduit extending through the aperture and at least a portion of the channel, the conduit having an inlet positioned in the chamber proximate to the distal end region of the fluid impermeable barrier.

13. The fluid collection device of claim 12, further comprising a shape memory polymer or metal having two arms extending longitudinally through the body with the channel positioned between the two arms.

14. The fluid collection device of claim 13, wherein the body includes two die-cut portions each at least partially defining the channel and having the two arms positioned between the two die-cut portions.

15. The fluid collection device of claim 11, wherein the front region and the rear region are welded together on a seam that extends from the aperture to the distal end region.

16. The fluid collection device of claim 11, wherein the front region and the rear region are welded together on a distal seam and a proximal seam with at least a portion of the fluid impermeable barrier between the distal seam and the proximal seam being void of a welded seam.

17. The fluid collection device of claim 11, wherein the body includes an extruded support and a fluid permeable membrane wrapped around the extruded support.

18. The fluid collection device of claim 17, wherein the fluid impermeable barrier includes a heat shrunk material.

19. A method of manufacturing a fluid collection device, comprising:
providing an elongated fluid impermeable barrier having a distal end region, a proximal end region, a first portion at least partially defining a chamber, an section in fluid communication with the chamber and positioned on the fluid collection device to receive urine discharged from a urethra at least proximate to the section, a second portion including a film, and an aperture sized and dimensioned to have urine drawn therethrough by a vacuum source;
positioning an elongated body that is at least partially fluid permeable at least partially within the chamber and extending at least partially across the section; and
bonding or welding the film of the second portion of the fluid impermeable barrier to the first portion of the fluid impermeable barrier with the elongated body positioned between the first portion and the second portion such that the first portion protrudes outward away from the second portion.

20-24. (canceled)

25. The method of claim 19, further comprising:
forming the first portion of the elongated fluid impermeable barrier with injection molding or die cutting foam to include a front region at least partially defining the chamber and the section;
wherein positioning an elongated body at least partially within the chamber includes positioning the elongated body at least partially within the chamber with a slot on the elongated body oriented away from the section on the front region of the fluid impermeable barrier and aligned with the aperture; and
after positioning the fluid permeable body at least partially within the chamber, bonding or welding the second portion of the fluid impermeable barrier including a fluid impermeable film to the front region opposite to the section with the film extending from the distal end region to the proximal end region of the fluid impermeable barrier and at least partially defining the chamber and the aperture.

26. The method of claim 25, further comprising laminating and/or die cutting the body.

27. The method of claim 25, further comprising positioning a conduit to extend through the aperture into the chamber at least partially in the slot of the body with an inlet of proximate to the distal end region of the fluid impermeable barrier.

28. The method of claim 25, further comprising positioning a shape memory polymer or metal proximate to the body.

29. The method of claim 19, wherein:
bonding or welding the film of the second portion of the fluid impermeable barrier to the first portion of the fluid impermeable barrier includes welding the first portion of the fluid impermeable barrier including a front region of plastic film to the second portion of the fluid impermeable barrier including a rear region of plastic film to at least partially define the chamber, the front region defining the section; and
positioning an elongated body at least partially within the chamber includes positioning the body within the chamber with a channel defined by the body aligned with the aperture.

30. The method of claim 29, further comprising inserting a conduit through the aperture and at least a portion of the channel to position an inlet on the conduit in the chamber proximate to the distal end region of the fluid impermeable barrier.

31. The method of claim 30, further comprising positioning a shape memory polymer or metal having two arms longitudinally in the body with the channel positioned between the two arms.

32. The method of claim 31, wherein positioning a shape memory polymer or metal having two arms longitudinally in the body includes positioning the shape memory polymer or metal having two arms longitudinally in the body between two separate die-cut fluid permeable portions of the body.

33. The method of 29, wherein welding a front region of plastic film to a rear region of plastic film includes welding the front region of plastic film to the rear region of plastic film on a seam that extends from the aperture to the distal end region.

34. The method of claim 29, wherein welding a front region of plastic film to a rear region of plastic film includes welding the front region of plastic film to the rear region of plastic film on a distal seam and a proximal seam, without welding at least a portion of the fluid impermeable barrier between the distal seam and the proximal seam.

35. The method of claim 29, further comprising extruding a support of the body and wrapping a fluid permeable membrane of the body around the extruded support.

36. The method of claim 29, further comprising heat shrinking the fluid impermeable barrier around at least a portion of the body.

37. The fluid collection device of claim 1, wherein the fluid collection device is curved longitudinally.
